(19) United States
(12) Patent Application Publication
(10) Pub. No.: US 2023/0352118 A1
(43) Pub. Date: Nov. 2, 2023

(54) Generative Modeling Leveraging Deep Learning for Antibody Affinity Tuning

(71) Applicant: Merck Sharp and Dohme LLC, Rahway, NJ (US)

(72) Inventors: Yara Seif, South San Francisco, CA (US); Xiao Xiao, Rahway, NJ (US); Kevin Teng, Rahway, NJ (US); Ming-Tang Chen, Rahway, NJ (US); Alan C. Cheng, Rahway, NJ (US); BoRam Lee, Rahway, NJ (US); Isha Verma, San Francisco, CA (US)

(21) Appl. No.: 18/311,138

(22) Filed: May 2, 2023

Related U.S. Application Data
(60) Provisional application No. 63/337,592, filed on May 2, 2022.

(51) Int. Cl. G16B 30/10; G16B 5/00

(52) U.S. Cl. CPC G16B 30/10; G16B 5/00

(57) ABSTRACT

A computer-implemented method is disclosed for candidate antibody exploration. The method includes receiving sequence reads from a sequencing system, wherein each sequence read comprises a target gene for expressing a candidate antibody, wherein each sequence read is associated with a binding affinity to a target antigen. The method includes generating a sequence representation for each sequence read, representing the amino acid sequences of the sequence read. The method includes training a binding affinity prediction model with the sequence representations and the binding affinities. The method includes generating a synthetic candidate sequence read that is different from the sequence reads. The method includes generating a sequence representation for the synthetic candidate sequence read. The method includes determining a binging affinity prediction for the synthetic candidate sequence read by applying the binding affinity prediction model to the sequence representation for the synthetic candidate sequence read.

### CROSS-REFERENCE TO RELATED APPLICATIONS

The present application claims the benefit of and priority to U.S. Provisional Application No. 63/337,592 filed on May 2, 2022, which is incorporated by reference in its entirety.

### BACKGROUND

### 1. Technical Field

The subject matter described relates generally to machine-learning and, in particular, to a deep learning approach to identifying antibodies that have desired binding properties.

2. Background Information

Antibodies and derived biologics are a major class of novel human therapeutics with over 70 FDA approvals in the past decade. In vitro display is one of the most common approaches for screening, selection and engineering of antibodies with desired affinity and specificity but is limited in size. Testing even a small fraction of the possible universe of antibodies is expensive and time consuming. Traditional methods of screening rely on biopanning over numerous iterations (e.g., more than five rounds). There is thus a need for more efficient ways to explore the sequence space to identify promising candidates for further study.

### SUMMARY

A method is disclosed for antibody affinity tuning utilizing a binding affinity prediction model and generative algorithm.

In one embodiment, to analytics system receives sequence reads relating to target genes used for expressing candidate antibodies in a biopanning screening. The biopanning screening utilizes one or more phage display, yeast display, or some combination thereof libraries of antibodies (e.g., single-chain antibodies). Fluorescence activated cell sorting (FACS) is used to sort the library against a target antigen. A set of candidate molecules are selected based on expression and binding profiles. The set of candidate molecules are sequenced using Next Generation Sequencing (NGS). In one embodiment, over 500,000 candidate single variable domain on a heavy chain (VHH) antibodies are sequenced.

The analytics system may preprocess the sequence reads in preparation for downstream analyses. The analytics system may translate the sequence reads into unique amino acid sequences describing the candidate antibodies assessed in the binding affinity assay. The analytics system may further encode the amino acid sequences into sequence representations, which are vectors of features.

The analytics system may train a binding affinity prediction model using the sequence representations and associated binding affinities. The binding affinity prediction model when trained inputs a sequence representation for an amino acid sequence read to output a predicted binding affinity against the target antigen. The analytics system may further calculate an interpretability score (e.g., including Shapley values) with the binding affinity prediction model to determine a contribution of each feature to the predicted binding affinity.

The analytics system may perform synthetic sequence generation using a mutation algorithm. The mutation algorithm may be informed by the sequence reads (or sequence representations), associated binding affinities (predicted by the binding affinity prediction model or quantified from a binding affinity assay), interpretability scores, or some combination thereof to generate one or more synthetic candidate sequence reads. The analytics system may predict the binding affinity for the synthetic candidate sequence reads using the binding affinity prediction model. The analytics system may perform an optimization algorithm to iteratively generate subsequent synthetic candidate sequence reads to optimize for binding affinity. The analytics system may select from the synthetic candidate sequence reads one or more optimal ones to use in a treatment for particular disease.

Implementing this method for antibody affinity tuning with the generative algorithm can quicken the antibody exploration process. Compared against traditional methods that utilized multiple rounds of biopanning screening, the analytical method utilizes sparse clinical data to predict binding affinity for synthetic candidate antibodies. This analytical method thus saves on clinical assaying resources and costs. Moreover, this analytical method advantageously can better explore the sequence space to assess many more candidate antibodies, prior to selection for use in treatment.

According to a first aspect of the disclosure, a computer-implemented method is disclosed comprising: receiving sequence reads from a sequencing system, the sequence reads associated with binding affinities to a target antigen, wherein each sequence read comprises a target gene for expressing a candidate antibody; generating sequence representations from the sequence reads, wherein each sequence representation is a feature vector representing the amino acid sequences of a corresponding sequence read; training a binding affinity prediction model with the sequence representations and the binding affinities, wherein the binding affinity prediction model is configured to input a sequence representation and to output a binding affinity prediction; generating a synthetic candidate sequence read that is different from the sequence reads; generating a sequence representation for the synthetic candidate sequence read; and determining a binding affinity prediction for the synthetic candidate sequence read by applying the binding affinity prediction model to the sequence representation for the synthetic candidate sequence read.

According to the first aspect, the computer-implemented method may further comprise: preprocessing the sequence reads, prior to generating the sequence representation for each sequence read.

According to the first aspect, preprocessing the sequence reads may comprise: demultiplexing the sequence reads by grouping matching sequences relating to one or more library indices for each of the sequence read into one sample.

According to the first aspect, preprocessing the sequence reads may comprise: removing one or more sequences pertaining to a primer used in sequencing of the target gene.

According to the first aspect, preprocessing the sequence reads may comprise: identifying, for each sequence read, sequences relating to one unique molecule identifier of a plurality of unique molecule identifiers; grouping sequence reads of similar length and having the same unique molecule identifier in a bin; and collapsing the sequence reads in each bin into a consensus sequence read.

According to the first aspect, preprocessing the sequence reads may comprise assembling each sequence read from a forward sequence read and a reverse sequence read of one nucleic acid molecule.

According to the first aspect, preprocessing the sequence reads may comprise: translating a sequence read from nucleotide sequences into amino acid sequences.

According to the first aspect, the binding affinity prediction model may be a machine-learning model.

According to the first aspect, the machine-learning model may be one of a deep-learning neural network, a convolutional neural network, or a residual neural network.

According to the first aspect, generating the synthetic candidate sequence read may be based on randomization.

According to the first aspect, generating the synthetic candidate sequence read may comprise: identifying a base sequence read with a high binding affinity; and mutating one or more amino acid sequences of the base sequence read.

According to the first aspect, the computer-implemented method may further comprise: calculating an interpretability score for each sequence representation that indicates the contribution of each feature in a given sequence representation to the predicted binding affinity for the given sequence representation.

According to the first aspect, the interpretability score comprises a Shapley value for each feature of the sequence representation.

According to the first aspect, the computer-implemented method may further comprise: determining the synthetic candidate sequence read is fit for a treatment of a particular disease if the binding affinity prediction for the synthetic candidate sequence read is above a threshold.

According to the first aspect, the computer-implemented method may further comprise: instructing a manufacturing system to manufacture the treatment for the particular disease comprising antibodies expressed from the synthetic candidate sequence read.

According to the first aspect, the computer-implemented method may further comprise: performing an optimization algorithm comprising iteratively: generating a subsequent synthetic candidate sequence read, determining a binding affinity prediction for the subsequent synthetic candidate sequence read by applying the binding affinity prediction model to a sequence representation for the subsequent synthetic candidate sequence read, and evaluating whether the binding affinity prediction improves upon a prior binding affinity prediction for a prior synthetic candidate sequence read.

According to a second aspect of the disclosure, a non-transitory computer-readable storage medium is disclosed storing instructions that, when executed by one or more computer processors, cause the one or more computer processors to perform the computer-implemented method according to the first aspect.

According to a third aspect of the disclosure, a system is disclosed comprising one or more computer processors and the non-transitory computer-readable storage medium according to the second aspect.

According to a fourth aspect of the disclosure, a treatment for treating a particular disease is disclosed comprising one or more antibodies manufactured according to the computer-implemented method according to the first aspect.

### BRIEF DESCRIPTION OF THE DRAWINGS

FIG. 1 is a block diagram of a networked computing environment suitable for providing antibody affinity tuning, according to one or more embodiment.

FIG. 2 is an overall workflow for discovering antibodies with desired binding properties utilizing a generative model, according to one or more embodiments.

FIG. 3 is a block diagram of the sequencing system of FIG. 1, according to one or more embodiments.

FIG. 4 illustrates sequencing of antibodies from a biopanning screening, according to one or more example implementations.

FIG. 5 is a block diagram of the analytics system of FIG. 1, according to one or more embodiment.

FIG. 6 illustrates processing of sequence reads from a biopanning screening, according to one or more example implementations.

FIG. 7 illustrates generating synthetic sequences leveraging a binding affinity prediction model to interpret binding affinities, according to one or more embodiments.

FIG. 8 is a flowchart of a method of generating synthetic sequences, according to one or more embodiments.

FIG. 9 is a block diagram illustrating an example of a computer, according to one or more embodiment.

The figures and the following description describe certain embodiments by way of illustration only. One skilled in the art will readily recognize from the following description that alternative embodiments of the structures and methods may be employed without departing from the principles described. Wherever practicable, similar or like reference numbers are used in the figures to indicate similar or like functionality. Where elements share a common numeral followed by a different letter, this indicates the elements are similar or identical. A reference to the numeral alone generally refers to any one or any combination of such elements, unless the context indicates otherwise.

### DETAILED DESCRIPTION

### Overview

The invention described provides for improvements to the field of antibody discovery leveraging a binding affinity prediction model to interpret binding properties of candidate antibodies. In one embodiment, an analytics system works with a sequencing system to input results from a biopanning screening. The biopanning screening utilizes yeast display to evaluate binding properties for a set of yeast libraries targeting a specific target antigen. The yeast libraries include candidate antibodies. The analytics system receives, from the sequencing system, sequence reads of one or more candidate antibodies in the biopanning screening process and binding affinities determined from the biopanning screening. The analytics system trains a binding affinity prediction model utilizing the sequence reads and the binding affinities, wherein the binding affinity prediction model predicts binding affinity for a sequence read pertaining to a candidate antibody. The analytics system may further implement an interpretability module to interpret how residues in the sequence read contribute to binding affinity. With the interpretability of which residues contribute to binding affinity, the analytics system may generate synthetic sequences to determine optimal candidate antibodies that optimize binding properties to the target antigen. Use of the binding affinity prediction model and the interpretability module in generating and evaluating synthetic sequences is advantageous over traditional biopanning processes by quickening exploration and discovery with sparse biopanning data.

As used herein “antibody” refers to a protein molecule capable of binding to an antigen. An antibody includes a chain of amino acids. Antibodies include monoclonal antibodies and polyclonal antibodies.

An “antigen” refers to a molecule that may be foreign to an individual. Antigens generally trigger an immune response in an individual.

“Binding affinity” refers to an attraction strength of two molecules, e.g., between an antibody and an antigen.

A “residue” refers to a subset of the amino acids in the chain, wherein residues may subdivide the chain of amino acids.

A “monoclonal” antibody has a monovalent affinity, i.e., binding to the same epitope of an antigen. The “monoclonal” antibody may comprise a variable portion and an invariable portion.

A “synthetic antibody” refers to an antibody that is synthetically generated, i.e., not originating from any individual.

A “binding affinity assay” refers to a clinical assay to evaluate binding affinity between molecules.

A “biopanning screening” refers to a process of evaluating binding affinity (e.g., performing a binding affinity assay) with a library of many antibodies (e.g., a yeast display, or a phage display) against a target antigen. A yeast display utilizes yeast cells to express antibodies on a cell surface from antibody genes, e.g., fused to the C-terminus of the Sachromyces cell surface protein Agall. Phage display utilizes phage a bacteriophage to express antibodies on a cell surface from antibody genes.

A “sequence read” or “sequence” represents an ordered set of monomers in a molecule as determined by a sequencing system. In the case of a protein molecule, the sequence read for the protein molecule refers to the ordered set of amino acids in the protein molecule. The sequence read may further include sequence portions relating to other molecules tagged onto the protein molecule during sequencing by the sequencing system. In the case of a nucleic acid molecule, the sequence read for the nucleic acid molecule refers to the ordered set of nucleotides in the nucleic acid molecule.

A “synthetic sequence read” or “synthetic sequence” refers to a synthetically generated sequence read, i.e., a synthetically generated ordered set of amino acids.

A “sequence representation” refers an encoding representing a sequence read, e.g., for a protein molecule or a portion thereof. The sequence representation may encode, in addition to the sequence read, additional properties of the sequence read. Additional properties may include length of the sequence read, protein folding structure, other physical properties of the protein molecule, other chemical properties of the protein molecule, or some combination thereof.

A “sequencing library” refers to a set of one or more unique sequences that is tagged onto all molecules in a sample for sequencing. Multiple sequencing libraries may be utilized for multiplexed sequencing of multiple samples.

A “unique molecule identifier,” or “UMI” refers to a unique sequence tagged onto a molecule in a sample. Generally, a set of UMIs are used to bin sequence reads into unique molecules in the sample.

### System Environment for Antibody Affinity Tuning

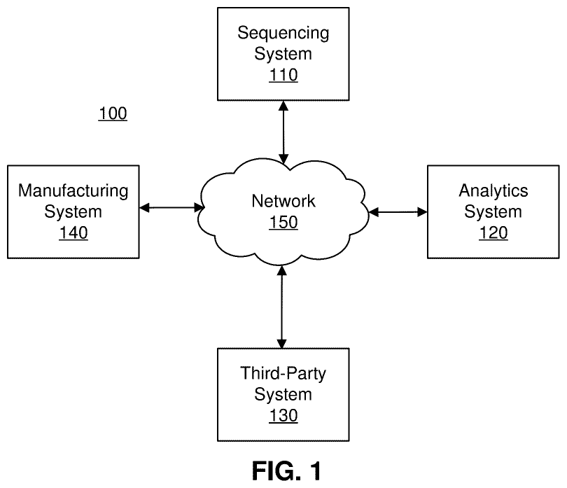

FIG. 1 illustrates one embodiment of a networked computing environment 100 suitable for providing antibody affinity tuning. In the embodiment shown, the networked computing environment 100 includes a sequencing system 110, an analytics system 120, a third-party system 130, and a manufacturing system 140, connected via a network 150. In other embodiments, the networked computing environment 100 includes different or additional elements. In addition, the functions may be distributed among the elements in a different manner than described. For example, in one configuration, all of the functionality described below is provided by a single computing system, which may or may not be connected to a network 150.

The sequencing system 110 analyzes antibodies or other biological molecules to determine amino acid or nucleotide sequences. In one embodiment, the sequencing system 110 includes a NGS system configured to sequence single-chain antibodies. The sequencing system 110 may sequence a set of candidate single-chain antibodies to generate corresponding sequences. The sequencing system 110 may utilize UMIs to increase the sequencing depth obtained by the sequencing system 110. The sequencing system 110 provides the sequence reads of the single-chain antibodies to the analytics system 120 for processing.

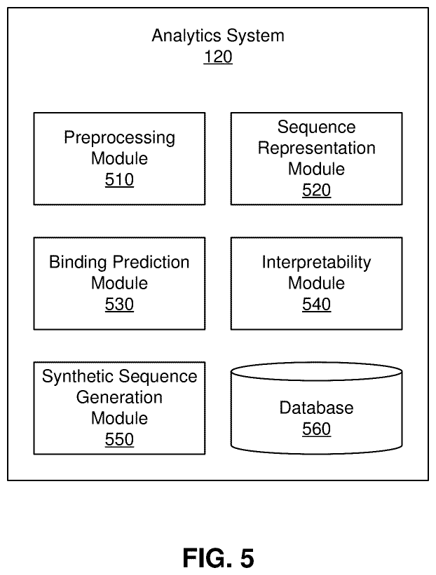

The analytics system 120 analyzes the sequence reads generated by the sequencing system 110 to identify strong candidates for further study. The analytics system 120 may be a computer configured to perform the various analyses. The analyses may include processing of sequence reads, representing sequence reads, training a binding affinity prediction model, deploying a binding affinity prediction model, interpreting a binding affinity of a candidate antibody, scoring residues or motifs of candidate antibodies, generating a synthetic sequence read, optimizing synthetic sequence reads, or some combination thereof. In various embodiments, the analytics system 120 may implement one or more machine-learning algorithms (e.g., deep neural networks, convolutional neural networks, residual neural networks, autoencoders, multi-layer perceptrons, decision trees, regressions, support-vector machine, etc.) to train one or more models, e.g., to train the binding affinity prediction model that predicts binding affinity for a sequence read for a candidate antibody to a target antigen. The operations of the analytics system 120 are further described in conjunction with FIGS. 5-7. The analytics system 120 may perform antibody affinity tuning to optimize candidate antibodies that are predicted to have high binding affinity to a target antigen. The optimal candidate antibodies may be provided to the manufacturing system 140 for full-scale manufacturing.

The third-party system 130 may store information relevant for antibody affinity tuning. The third-party system 130 may be a computer or a database storing the relevant information. Example information relevant to antibody affinity tuning may include prior antibody sequences, corresponding binding affinities, antibody residues, folding structure, other physical properties of prior antibody sequences and/or residues, chemical properties of prior antibody sequences and/or residues, other information related to antibodies and/or antigens, or some combination thereof.

The manufacturing system 140 manufactures antibodies. The manufacturing system 140 may include devices used in the manufacturing of antibodies. For example, the manufacturing system may manufacture nucleic acid sequences to be expressed with a cell-line as the antibodies. The manufacturing system may also include a bioreactor that cultivates the cell-line expressing the antibodies with the nucleic acid sequences. The manufacturing system 140 may further lyse the cell-line and isolate the expressed antibodies. The manufacturing system 140 may further purify the isolated antibodies. The manufacturing system 140 may perform a binding affinity assay to assess binding affinity of produced antibodies. The binding affinity assay may be targeted with one or more antibodies, or may be part of a biopanning screening.

The network 150 provides the communication channels via which the other elements of the networked computing environment 100 communicate. The network 150 can include any combination of local area and wide area networks, using wired or wireless communication systems. In one embodiment, the network 150 uses standard communications technologies and protocols. For example, the network 150 can include communication links using technologies such as Ethernet, 802.11, worldwide interoperability for microwave access (WiMAX), 3G, 4G, 5G, code division multiple access (CDMA), digital subscriber line (DSL), etc. Examples of networking protocols used for communicating via the network 150 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 150 may be represented using any suitable format, such as hypertext markup language (HTML) or extensible markup language (XML). In some embodiments, some or all of the communication links of the network 150 may be encrypted using any suitable technique or techniques.

### Workflow for Antibody Production

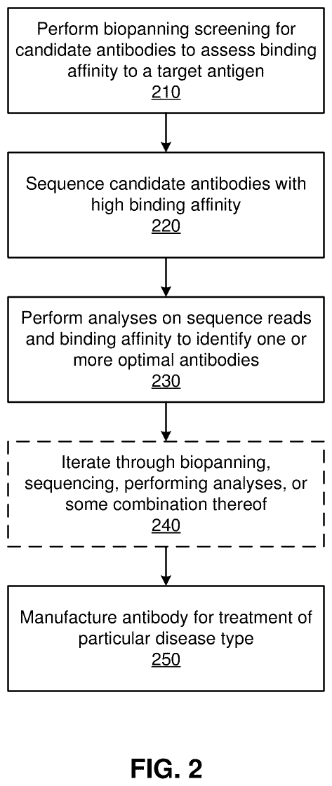

FIG. 2 is an overall workflow 200 for discovering antibodies with desired binding properties utilizing a generative model, according to one or more embodiments. Steps of the overall workflow 200 may be performed by one or more of the components of the networking computing environment 100 of FIG. 1. A user may further provide input and/or control the one or more components of the networking computing environment 100 of FIG. 1 in the overall workflow 200.

The overall workflow 200 includes performing 210 a biopanning screening for candidate antibodies to assess binding affinity to a target antigen. The biopanning screening includes preparation of a yeast library, a phage library, or some combination thereof, each library including candidate antibodies. The candidate antibodies may be generated by a manufacturing system (e.g., the manufacturing system 140). The sequences for the candidate antibodies may be created using a randomization technique. In other embodiments, the sequences for the candidate antibodies may be generated as synthetic sequences by an analytics system (e.g., the analytics system 120). The libraries are screened against a target antigen. For example, a microtiter plate coated with a target antigen is used to bind the candidate antibodies. The microtiter plate may be washed to remove unbound candidate antibodies. In some embodiments, the washing process may further grade the binding strength of the candidate antibodies. After the washing process, the bound candidate antibodies may be eluted from the microtiter plate for sequencing of the candidate antibodies. In some embodiments, the manufacturing system 140 performs the biopanning to assess binding affinity.

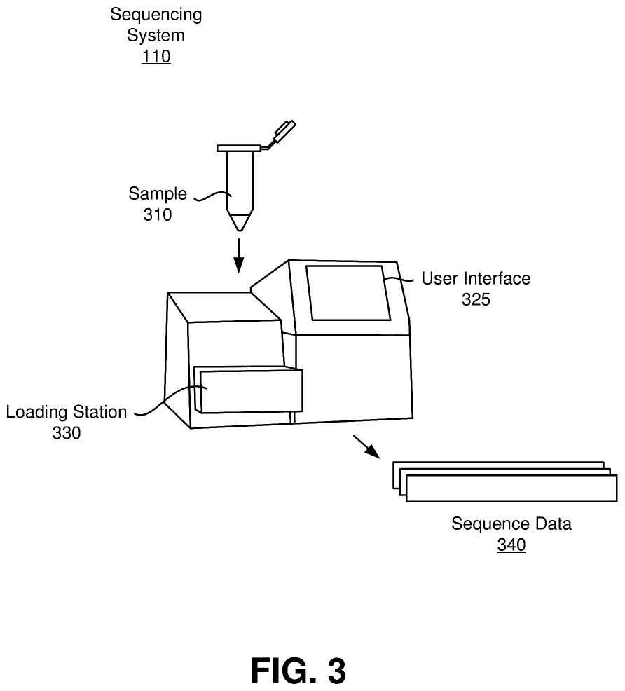

The overall workflow 200 includes sequencing 220 the candidate antibodies, e.g., antibodies with high binding affinity as assessed in a binding affinity assay or a biopanning screening. The candidate antibodies may be associated with binding affinity according to a binding affinity assay. As another example, candidate antibodies with a high binding affinity may be selected from the biopanning screening. Sequencing of the candidate antibodies may involve utilizing a sequencing library with one or more indices. The sequencing library aids in multiplex sequencing of multiple samples in a sequencing device. Each index (also referred to as “library tag”) in a sequencing library is a unique set of molecules that can be ligated onto another molecule. For example, the index may be a unique sequence of amino acids that can be ligated onto a protein molecule. Sequencing may also involve ligating, to each protein molecule in a sample, a plurality of unique molecule identifiers (UMIs). Each UMI may be distinct from other UMIs. The likelihood that two distinct protein molecules that coincidentally have the same amino acid sequence and are ligated with the exact same UMI is extremely low. Thus, having two sequence reads with the same amino acid sequence with the same UMI is unlikely to be due to two initially distinct protein molecules, and rather protein molecules that may be replicates of one another, e.g., during an amplification process in the sequencing. Once prepped, a sequencing system (e.g., the sequencing system 110) sequences a protein molecule by detecting an ordered set of amino acids present on the protein molecule. yielding a sequence read for each protein molecule. In some embodiments, sequencing may further involve amplification of protein molecules, thereby increasing sequencing depth. The improved sequencing depth improves capture of protein molecules present in a sample. Further details relating to sequencing are described below in conjunction with FIGS. 3 & 4.

The overall workflow 200 includes performing 230 one or more analyses on the sequence reads and binding affinities to identify one or more optimal antibodies. The analytics system 120 may perform the one or more analyses. In general, the one or more analyses may be part of antibody affinity tuning. In one or more embodiments, analyses include processing the sequence reads, generating a sequence representation for each sequence read, training and/or deploying a binding affinity prediction model using the sequence reads, calculating an interpretability score to interpret a predicted binding affinity, generating synthetic sequence reads, evaluating synthetic sequence reads, exploring sequence space to identify optimal synthetic sequences, or some combination thereof. Further details relating to the one or more analyses is described in conjunction with FIGS. 5-7.

The overall workflow 200 includes iterating 240 through biopanning screening, sequencing, performing one or more analyses, or some combination thereof. For example, the analytics system 120 may provide synthetic sequence reads pertaining to candidate antibodies to be assessed for binding affinity in a binding affinity assay to the manufacturing system 140. The manufacturing system 140 may generate one or more candidate antibodies using the synthetic sequence reads. The manufacturing system 140 may further perform the binding affinity assay with the candidate antibodies to assess binding affinity of the candidate antibodies. The sequencing system 110 may sequence the candidate antibodies from the binding affinity assay and provide the sequence reads to the analytics system 120. The analytics system 120 may perform additional analyses using the sequence reads and the binding affinities. For example, the analytics system 120 may validate and/or update a trained binding prediction model using the sequence reads and the binding affinities. The analytics system 120 may further use the sequence reads and the binding affinities to inform exploration of the sequence space when performing antibody affinity tuning.

The analytics system 120 may utilize one or more criteria in determining the optimal candidate antibodies. For example, the analytics system 120 may select one or more optimal candidate antibodies that have maximum binding affinities (e.g., as predicted by a trained binding affinity prediction model or as assessed by a binding affinity assay) and with diverse sequence reads. Another example selection criterion avoids selection of sequences that include cysteine in the complementarity determining regions (CDR) domain, e.g., which poses a risk of creating a disulfide bond.

The overall workflow 200 includes manufacturing 250 an antibody for treatment of a particular disease type. The treatment may comprise one or more antibodies identified as optimal candidate antibodies through the process of antibody affinity tuning. The manufacturing system 140 may manufacture the antibodies for the treatment. Manufacturing may include growing a cell-line, infecting the cell-line with genetic material coded for expressing the optimal candidate antibodies, cultivating the cell-line to express the antibodies, isolating the expressed antibodies, or some combination thereof. The manufactured treatment is ready for dosing and use by a patient for the treatment of the particular disease type.

### Sequencing System

FIG. 3 is an exemplary sequencing system 110 for sequencing protein molecules, in accordance with one or more embodiments. The sequencing system 110 may comprise additional, fewer, or different components than those listed herein FIG. 3.

In various embodiments, the sequencing system 110 receives a sample 310. In a protein sequencing application, the sample 310 may include one or more protein molecules to be sequenced. In a nucleic acid sequencing application, the sample 310 may include one or more nucleic acid molecules to be sequenced. As shown in FIG. 3, the sequencing system 110 can include a graphical user interface 325 that enables user interactions with particular tasks (e.g., initiate sequencing or terminate sequencing) as well as one more loading stations 330 for loading a sequencing cartridge including the sample 310 and/or for loading other materials for performing the sequencing assays. Additional materials may include library indices, UMIs, buffers, other chemicals for use in the sequencing assay, or some combination thereof. Therefore, once a user of the sequencing system 110 has provided the necessary reagents and sequencing cartridge to the loading station 330 of the sequencing system 110, the user can initiate sequencing by interacting with the graphical user interface 325 of the sequencing system 110. The sequencing system 110 may further comprise fluidic channels for directing reagents appropriately throughout the sequencing system 110. The sequencing system 110 may also comprise one or more detectors configured to read out sequences from the one or more molecules. The detectors may be photosensors configured to read out differential light signatures corresponding to the type of sequence present in a molecule. Once initiated, the sequencing system 110 performs the sequencing and outputs sequencing data 340 including the sequence reads of the enriched fragments from the sample 310. In other embodiments, the sequencing system 110 may omit the user interface and may receive instructions via another computing device, e.g., via a network. In some embodiments, the sequencing system 110 is communicatively coupled with an analytics system (e.g., the analytics system 120) to provide the analytics system with the sequencing data 340 by the sequencing system 110.

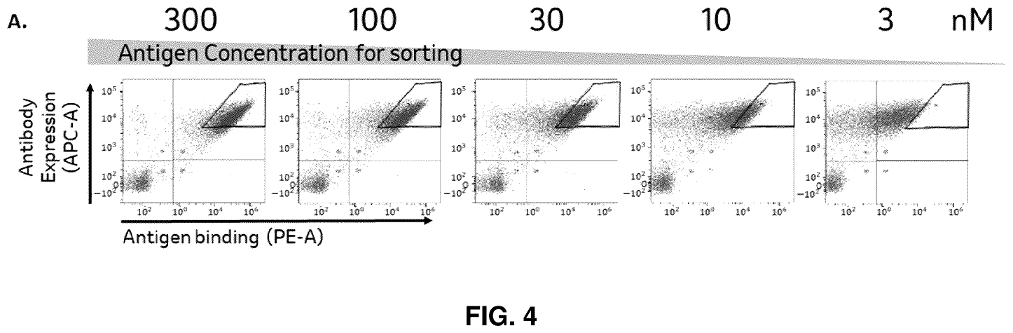

FIG. 4 illustrates sequencing of antibodies from a biopanning screening, according to one or more example implementations. The sequencing may be performed by the sequencing system 110 of FIG. 1.

FIG. 4 is illustrative of embodiments including generation, processing, and analysis of large next generation sequencing data for 5.6×10\hat{ }5 UMI-tagged single-chain antibodies sorted against MERS at five distinct antigen concentrations: A) Flow cytometry output plots following FACS selection at five antigen concentrations. Each binding affinity assay targets a different concentration of a target antigen. The plot for each binding affinity assay plots each candidate antibody based on the candidate antibodies binding affinity (plotted as “antigen binding” on the x-axis) against expression of the antibody (plotted as “antibody expression” on the y-axis). The candidate antibodies above a threshold expression and above a threshold binding affinity can be sequenced for analyses. The binding affinities of the candidate antibodies may be quantified based in part on the target antigen concentration. For example, candidate antibodies (above a threshold expression and above a threshold binding affinity) that bound to the target antigen at the lowest concentration (e.g., 3 nM) would have a stronger binding affinity than candidate antibodies (above a threshold expression and above a threshold binding affinity) that bound to the target antigen at a higher concentration (e.g., 10, 30, 100, or 300 nM).

The biopanning screening leveraged the Humanized-2AA framework region as a scaffold. Humanized-2AA is a modified version of the human framework IGHV3-23*04 at two positions 49(G ->Q) and 50 (L->R). The framework region IGHV3-23*04 is the closest human homolog to the alpaca IGHV3S53 scaffold, which is predominant across large next generation sequencing datasets. Residues Q49 and R50 have been previously shown to be critical to VHH stability. The biopanning further leveraged available alpaca antibody sequences and analyzed the amino acid profile obtained from a multiple sequence alignment to identify positions of low conservation in the complementarity determining regions. As a result, three positions in CDRH1 were prioritized—namely positions 30, 31 and 34—as well as five positions in CDRH2—namely, positions 49, 51A, 52, 54 and 55—for diversification. Because of its dominant contribution to binding, the entirety of the CDRH3 loop was also prioritized for diversification. The amino acid length was set to match the natural camelid repertoire which ranged between 6 and 18 residues.

In the example implementation, a biopanning screening creates a display campaign targeting the MERS antigen and subdivided the output into three distinct VHH libraries each spanning a sub-range in loop length: 6-10 (C1), 11-14 (C2) and 15-18 (C3) amino acids. For each library, spiked nucleotide ratios bias towards germline codons on a positional basis, and trinucleotide mutagenesis (TRIM) to synthesize the loops. The library may exclude stop codons, cysteine, methionine, and tryptophan residues. Synthesized CDRH3 fragments were then spliced onto the Humanized-2AA framework region using a three-step PCR overlap extension. Diversity was then introduced within the CDRH1 and CDRH2 domains, and the assembled VHH constructs were transformed in yeast for display and selection. Each library was evaluated for expression and binding using FACS against 4 different antigen concentrations (3 nM, 10 nM, 30 nM, 100 nM, and 300 nM). Cells sorted at higher concentrations correspond to weaker binders while cells sorted at lower antigen concentrations are the strongest binders. The binding affinity threshold obtained anywhere between 102 and 106 cells that fell within the gate of interest in each sample. The cells that were captured were extracted for plasmids and ran a single-cycle PCR to attach a 22 base pair long library tag, a 16 base pair long unique molecular identifier, and a 23 base pair long PCR primer to the 5′ end.

### Analytics System

FIG. 5 is a block diagram of the analytics system 120, in accordance with one or more embodiments. In the embodiment shown, the analytics system 120 includes a preprocessing module 510, a sequence representation module 520, a binding prediction module 530, an interpretability module 540, a synthetic sequence generation module 550, and a database 560. In other embodiments, the analytics system 120 includes additional, fewer, or different components than those listed herein. In addition, the functions may be distributed among the elements in a different manner than described.

The preprocessing module 510 preprocesses sequencing data received from the sequencing system 110 to prepare it for analysis. In one or more embodiments, the preprocessing pipeline includes a combination of the following: (1) adapter and read trimming; (2) read assembly; (3) UMI binning; (4) consensus sequence generation; (5) sequence translation; (6) de-duplication; and (7) length and sequence similarity filtering.

In one embodiment, the preprocessing module 510 performs adapter and read trimming. In performing adapter and read trimming, the preprocessing module 510 may demultiplex sequence reads into corresponding sequencing libraries based on library indices present on the sequence reads. The preprocessing module 510 may further identify sequences relating to a primer, sequences relating to an UMI, sequences relating to tail ends. For example, the preprocessing module 510 detects at the 5′ end a library index (or referred to as a “library tag”). The various library indices may be known by the analytics system 120, such that the preprocessing module 510 may search for the library index present in the same position on each of the sequence reads. and reads in which no primers are detected are discarded. The preprocessing module 510 may also extract sequences relating to the UMI. In embodiments with library indices tagged to the 5′ end, the preprocessing module 510 may trim sequences at the 3′ end. The preprocessing module 510 may further discard sequence reads according to one or more fitness criteria. In a first example fitness criterion, the preprocessing module 510 may also evaluate a Phred quality cutoff (Q) (e.g., 10, 15, or 20) for determining whether sequence reads are sufficiently noise-free. In another example, a fitness criterion requires sequence reads to be of a minimum length (e.g., 30, 33, 36, 39, 42, or 45 bp). A third example fitness criterion requires sequence reads to include one or more of: a primer, a library index, and an UMI. Sequence reads missing any of the required portions can be deemed unusable.

The preprocessing module 510 may further assemble the sequence reads, e.g., in preparation for analyses by other components. The preprocessing module 510 may assemble a sequence read as comprising a target portion relating to the molecule sequenced from the sample paired with a UMI. In one or more embodiments, the preprocessing module 510 may assemble sequence reads by combining sequence read pairs, for a forward read and a reverse read. The preprocessing module 510 may require a minimum overlap between sequence read pairs (e.g., 6, 8, 10, 12, 14, 16, 18, or 20).

The preprocessing module 510 may further bin the sequence reads together according to the UMIs. For example, the preprocessing module 510 may cluster sequence reads together based on similarities between two sequence reads and further based on matching UMI sequences. For example, similarities considered include, but are not limited to, length of sequence reads, distance between sequence reads (e.g., a count of differences in sequences between two sequence reads), a minimum overlapping portion, etc. The preprocessing module 510 may collapse a bin of sequence reads into a consensus sequence read, for use in further analyses. Collapsing of sequence reads in a bin may comprise identifying, for each position, a consensus sequence among the sequence reads.

In one or more embodiments, the preprocessing module 510 may translate nucleic acid sequence reads into a protein sequence. The preprocessing module 510 may utilize a frame of six sequences to determine an amino acid coded for by the nucleic acid sequence read. The preprocessing module 510 evaluates the nucleotide sequences with the frame and outputs the longest, uninterrupted protein sequence. For each sample (e.g., the output of a library screened against a single antigen concentration), the preprocessing module 510 may further de-duplicate protein sequences. The preprocessing module 510 may further select (e.g., at random) a number (e.g., one hundred) of sequences as a representative sampling of the entire dataset.

In one or more example implementations, the preprocessing module 510 may translate the nucleic acid sequences into protein sequences, informed by other information on known antibodies. For example, the preprocessing module 510 may embed the sequence reads using the ProtVec framework, may group the sequence reads into clusters using the MiniBatchKMeans command. According to such example implementations, the preprocessing module 510 may further set initialization to random, with the number of initializations set to ten, and the maximum number of iterations set to three hundred. The preprocessing module 510 may further align sequence reads (e.g., the representative sampling) in the Molecular Operating Environment (MOE) and antibody domains annotated using the Chemical Computing Group numbering scheme ((CCG) 2021). This seed alignment may be used to build an HMM profile (hmmbuild) and the assembled sequences may be aligned using HMMer (hmmalign). The preprocessing module 510 may, alternatively, align assembled sequences according to a known framework. The preprocessing module 510 may further filter out any sequence read missing any one of the three complementarity domain regions. If amino acid sequences are observed across more than one class within the same target group, the preprocessing module 510 may re-assign the amino acid sequence to the highest fitness class (e.g., the lowest antigen concentration). In effect, the fitness class is a discrete qualitative measure of the binding affinity of the amino acid sequence (or candidate antibody).

The sequence representation module 520 converts the sequences generated by the sequencing system 110 (before or after preprocessing) into a sequence representation. A sequence representation is suited to use with one or more machine-learning models. The sequence representation used can have a significant impact on the accuracy of the predictions generated by the machine-learning model.

In one embodiment, three different aspects of sequence representation are considered before use with the machine-learning model: (1) sequence domains; (2) padding; and (3) embedding. Single variable domain on a heavy chain (VHH) antibodies are subdivided into seven domains, with the three complementarity determining regions (CDRs) being the largest contributors to binding energy. The library may be designed such that positions of diversification are restricted to CDRs to improve the hit rate for optimized binders. Thus, to avoid additional training time in domains which should theoretically not vary (e.g., the framework region domains) and to minimize model training on potential sequencing noise in the theoretically non-diversified regions, the machine-learning model input may, in one embodiment, be restricted to the concatenation of one or more of the three CDRs.

Because many types of machine-learning model expect inputs of equal length, the sequence representation module 520 may pad amino acid sequences to unify sequence lengths. Padding is a process by which sequences are artificially padded to the same length with zeros. Example padding strategies include the post, pre, mid, strf, ext, zoom, and an “aligned” (aln) strategy in which padding is positioned by the gaps which are placed by the Hidden Markov model (HMM) alignment algorithm.

In addition, because many types of machine-learning model can only consider numbers as inputs, different strategies may be adopted to convert VHH sequences into numbers. Because sequence representation has an impact on the performance of deep learning models, selection of embedding may be a significant factor in model performance. The following table illustrates eleven example embedding approaches.

TABLE 1

| | Embedding | | |
|---|---|---|---|
| Abstraction level | name | Nb. dimensions | Type |
| Directly | AA-index | 117 × 565 | Physico-chemical descriptors |
| Measured | VHSE-1 | 117 × 43 | Curated set of residue-level |
| | | | physicochemical descriptors |
| | One-hot | 117 × 21 | Residue specific descriptors |
| | encoding | | |
| Grouped based on | MOS | 117 × 7 | Residue classification based on their |
| direct | | | dipole and side chain volumes |
| measurement | | | |
| Adjusted principal | Physical3 | 117 × 3 | Curated descriptors to model |
| components (direct | | | hydrophobicity, volume and polarity |
| measurements) | | | |
| Principal | Z-scale | 117 × 5 | Top 5 PC scores from a set of 87 |
| | | | descriptors |
| components of | PC-11 | 117 × 11 | First 11 PCs of the matrix of the AA- |
| | | | index descriptors |
| large sets of direct | PC-18 | 117 × 18 | First 18 PCs of the matrix of the AA- |
| | | | index descriptors |
| measurements | VHSE-2 | 117 × 8 | Top 8 PC scores from VHSE-1 |
| Learnt embeddings | Prot Vec | 1 × 100 | Unsupervised deep learning of large |
| | | | protein data-sets (Word2Vec) |
| | ESM | 1 × 1,280 | Unsupervised deep learning of large |
| | | | protein data-sets (MSA Transformer) |

The representations included in table 1 include one-hot encoding (oh), directly measured per-residue physico-chemical descriptors (AA-index) or a curated subset thereof (VHSE-1), grouped residue classifications, a dimensionally reduced representation of the large physico-chemical descriptor sets (Physical3, Z-scale, PC-11, PC-18, and VHSE-2), and learnt embeddings (ProtVec and ESM). For any particular use case, the performance of the model may be evaluated during training using different embeddings using any suitable metric or metrics of model performance and the embedding that produces the best performing model as indicated by the metrics) selected. The sequence representation module 520 may further utilize hybrid representations combining two or more of the above embeddings.

The binding prediction module 530 implements a binding affinity prediction model to predict the binding strength of input sequences with a target antigen. To train the binding affinity prediction model, the binding prediction module 530 utilizes the sequencing data and corresponding binding affinity from the biopanning screening and sequencing. For example, to train the binding affinity prediction model, the binding prediction module 530 may input batches of sequence representations to predict binding affinity. The binding prediction module 530 may then train the binding affinity prediction model by tuning parameters or weights of the binding affinity prediction model to minimize a loss between the predicted binding affinity and the true binding affinity (e.g., as determined by the biopanning screening assay).

In various embodiments, the binding affinity prediction model is a machine-leaned model. Example machine-learning algorithms include neural networks, autoencoder, multi-layer perceptrons, decision trees, regressions, classifiers, support vector machine, derivatives thereof, or a combination thereof. A variety of architectures may be used, including multi-layer perceptrons (MLP), bi-directional long short-term memory models (bi-LSTM), convolutional neural networks (CNN), and residual convolutional neural networks (ResNet). In one embodiment, a CNN architecture is used that includes two convolutional layers, each followed by a maxpooling layer and a ReLU activation layer (e.g., or a leakyReLu activation layer), and three fully connected layers, each of which are also followed by a ReLU activation layer. The last fully connected layer may include four output channels (one for each class of output, e.g., mapped to antigen concentration in the FACS). Principal component may be used as the embedding type with a batch size of 60 and a learning rate of 0.01.

In one embodiment, the binding prediction module 530 may optimize an architecture of the binding affinity prediction model. For example, with a neural network implemented binding affinity prediction model, the binding prediction module 530 may use a two-part optimization: (1) neural architecture search (NAS); and (2) hyperparameter optimization (HpOpt). Starting from a large number of potential architecture and hyperparameters, an iterative optimization may be used. For each cycle, the top N (e.g., five) performing architectures may be selected following NAS and HpOpt implemented for each. One or both search spaces (i.e., the NAS and HpOpt spaces) may be further tailored by introducing additional constraints or fixing variables according to the two optimization results. For example, for the first cycle, the hyperparameter values may be fixed to a previously derived set of values including the dropout fraction, number of epochs, number of batches, learning rate, momentum, padding, and embedding, etc.

In one example, a neural architecture search is performed for both types of basic blocks (ResNets and CNNs). Specifically, for the CNN, the number of convolutional and fully connected layers may be allowed to vary, along with the kernel size and count, dilation, activation layers, presence of batch normalization or max pooling layers, and the final loss functions used. Table 2 illustrates various configurations of CNN that may be used.

TABLE 2

| Architectural parameter | Search space type | Search space |
|---|---|---|
| Number of Convolutional | Choice (n = 6) | 1, 2, 3, 4, 5, 6 |
| layers | | |
| Output channels - Conv | Choice (n = 3) | 32, 64, 128 |
| Number of Fully | Choice (n = 3) | 1, 2, 3 |
| Connected layers | | |
| Output channels - FC | Choice (n = 3) | 2\hat{ }(5 + nb. FC + 1) |
| Kernel size | Choice (n = 10) | 2, 3, 4, 5, 6, 7, 8, 9, 10, 11, |
| | | 12 |
| Dilation | Choice (n = 5) | 1, 2, 4, 8, 16 |
| Activations | Choice (n = 4) | SeLU, ReLU, 1ReLU, tanh |
| Final activation layer | Choice (n = 4) | LogSoftmax, Softmax, |
| | | ReLU |
| Loss function | Choice (n = 2) | Weighted Focal Loss |
| | | (WFL) with gamma = |
| | | choice(1, 2, 3, 4, 5), Cross |
| | | Entropy Loss (CEL) |

In example implementations with residual neural networks (ResNets), the binding prediction module 530 may further tune the number of ResNet basic blocks along with the architecture (i.e., the order of layers, bottleneck, and dilation), the kernel size of the first layer, the number of residuals per basic block, and the type of activation layer. The Hyperopt package may be used to explore the architectural search space of both model types selecting the Tree Parzen Estimator as the algorithm to optimize for accuracy on the test set, and allowing up to 100 trials. Table 3 illustrates some example ResNet configurations.

TABLE 3

| Architectural parameter | | Search space type | Search space |
|---|---|---|---|
| Block 1 | Nb. Residuals | Choice (n = 4) | {2, 3, 4, 5} |
| | Dilation | Choice (n = 2) | {1, 2} |
| Block 2 | Nb. Residuals | Choice (n = 6) | {2, 3, 4, 5, 6, 7} |
| | Dilation | Choice (n = 3) | {1, 2, 4} |
| Block 3 | Nb. Residuals | Choice (n = 6) | {2, 3, 4, 5, 6, 7} |
| | Dilation | Choice (n = 3) | {1, 2, 4} |
| Block 4 | Nb. Residuals | Choice (n = 4) | {2, 3, 4, 5} |
| | Dilation | Choice (n = 4) | {1, 2, 4, 8} |

In some embodiments embodiment implementing a convolutional neural network (CNN), the binding prediction module 530 may toggle the number of convolutional layers and fully connect layers being allowed to vary, along with the kernel size, the number of kernels, the activation type, the dilation size, or some combination thereof. Strides may be fixed at 1, while dilations may be allowed to vary so as to allow for increased resolution of feature maps at deeper layer without sacrificing the size of the receptive field. Hyperopt may be used to automate the neural architecture search and run a predetermined number of times (e.g., fifty) with these settings using the tree parzen estimator as the optimization algorithm and the accuracy on the test set as the performance metric.

In various embodiment, the hyperparameter search space includes using various numbers of embeddings. Additional hyperparameters may include the learn rate, momentum, and batch size. Table 4 illustrates some example hyperparameter ranges.

| | Search space | |
|---|---|---|
| Hyperparameter | type | Search space |
| Embedding | Choice (n = | {AA-index, VHSE-1, VHSE-2, One-hot, MOS, |
| | 11) | Physical3, Z-scale, PC-11, PC-18, Prot-Vec, |
| | | ESM} |
| Padding | Choice (n = 7) | {aln, pre, mid, post, strf, zoom, ext} |
| Learn rate | Uniform | 0.005-0.01 |
| Batch size | Choice (n = 3) | {300, 400, 500} |
| Momentum | Uniform | 0.85-0.99 |
| Weight decay | Uniform | 0.0001-0.01 |
| WFL alpha | Uniform | 0.01-0.055 |
| WFL gamma | Choice | {1, 2, 3, 4, 5} |
| Dropout fraction | Uniform | 0.5-1 |

In one embodiment, hyperopt is used to explore the hyperparameter search space, allowing a predetermined number (e.g., one hundred) hyperparameter sets to be tried. For CNNs, max pooling may be used right after the convolutional layer and preceding the activation step. The initialization may be used for the weights, training the network de novo, and using stochastic gradient descent, with a preset weight decay (e.g., 0.001). The number of epochs may be capped (e.g., at 300) while implementing early stopping, in which training is stopped whenever the validation rate stops decreasing for a predetermined number (e.g., five or ten) consecutive epochs. Typically, the predetermined number of epochs is less for ResNet architectures than CNNs.

Once a model is trained by the binding prediction module 530, the binding prediction module 530 may apply the binding affinity prediction model to a candidate antibody sequence representation to predict a binding affinity (also referred to as “binding affinity prediction”) of the candidate antibody. In some embodiments, the binding affinity prediction may be a discrete value selected from a discretized range (e.g., {1, 2, 3, 4, 5}) could refer to varying levels of binding strength, with 1 as the lowest and with 5 as the highest. In other embodiments, the binding affinity prediction may be a value selected from a continuous range (e.g., binding affinity selected from the range from 0 to 100).

The interpretability module 540 interprets the predicted binding affinity of a candidate antibody output by a binding affinity prediction model. The interpretability module 540 may determine an interpretability score for a predicted binding affinity for a candidate antibody's sequence representation. The interpretability score may indicate a contribution of each feature in the sequence representation to the predicted binding affinity. For example, for each feature in the sequence representation, the interpretability score may indicate whether the feature in the sequence representation positively, negatively, or negligibly to the predicted binding affinity. The interpretability score may further indicate a magnitude of a feature's contribution to the predicted binding affinity. In one or more embodiments, the interpretability module 540 determines the interpretability score as a Shapley value for each feature of the sequence representation.

The interpretability module 540 may generate the Shapley value for a given feature of a sequence representation by determining an average marginal contribution of the given feature. The interpretability module 540 creates a modulated sequence representation from a base sequence representation by modulating the given feature in the sequence representation. Modulating the given feature entails sampling a different value for the given feature, while holding other feature values constant. The interpretability module 540 may input the modulated sequence representation into the binding affinity prediction model to output a pseudo binding affinity prediction. The interpretability module 540 may determine the Shapley value based on a difference between the pseudo binding affinity prediction (from the modulated sequence representation) and the binding affinity prediction (from the original sequence representation). The interpretability module 540 may iteratively modulate the given feature in the sequence representation and then average the differences between the pseudo binding affinity predictions (from modulated sequence representations) and the binding affinity prediction (from the original sequence representation). The interpretability module 540 may generate a Shapley value for each feature in a sequence representation to quantify contribution of each feature to the binding affinity prediction.

The synthetic sequence generation module 550 receives one or more candidate sequence reads from the binding prediction module 530 that are predicted to have a high binding strength with the target. Various metrics may be used to select candidate sequence reads. For example, a quality score may be calculated by combining (e.g., multiplying) the predicted binding strength and the corresponding confidence score outputted by the machine-learning model. Regardless of the precise metric used, the candidate sequence reads are ranked and one or more are selected. The top N rated sequences may be selected (where N is a preselected integer which may be configurable) or any sequences for which the selected metric exceeds a threshold may be selected.

The synthetic sequence generation module 550 generates new, synthetic candidate sequence reads from the selected candidate sequence reads. In one embodiment, a mutation algorithm is applied to the selected candidate sequence reads to generate the synthetic candidate sequence reads. The synthetic sequences may in turn be provided as input to the binding affinity prediction model to generate a binding affinity prediction. In one or more embodiments, the mutation algorithm evaluates an initial set of candidate sequence reads to determine one or more synthetic sequences. The mutation algorithm may further evaluate an interpretability score for a candidate sequence. The synthetic sequence generation module 550 may iteratively apply the mutation algorithm to generate subsequent synthetic candidate sequence reads to optimize binding affinity. This process may be iterated until an end condition is met (e.g., a target binding strength is obtained or the predicted binding strengths stop increasing by at least a specified amount (or start decreasing), etc.

In one or more embodiments, the synthetic sequence generation module 550 may rank the candidate sequence reads and the synthetic candidate sequence reads based on predicted binding affinity. The synthetic sequence generation module 550 may further identify patterns in top ranked candidate sequence reads. The identified patterns may include information on positions in the candidate sequence reads that are highly contributing to binding affinity, information on residues that are highly contributing to binding affinity, information on features in the sequence representations that are highly contributing to binding affinity, other relevant patterns, or some combination thereof.

In other embodiments, the mutation algorithm may leverage additional clinical data. The analytics system 120 may provide the synthetic candidate sequence reads to be manufactured into antibodies (e.g., by the manufacturing system 140) and assessed for binding affinity in a binding affinity assay (e.g., by the manufacturing system 140). The results of the binding affinity assay may validate the predicted binding affinity of the synthetic candidate sequence reads. The mutation algorithm can leverage that validation to determine which mutations to keep and which mutations to forgo.

In one or more embodiments, the mutation algorithm may leverage machine-learning algorithms to optimize the synthetic candidate sequence reads generated by the mutation algorithm. For example, the mutation algorithm may rely on stochastic gradient descent to continue iteratively generating synthetic candidate sequence reads. The mutation algorithm may tune an exploration parameter, and/or an exploitation parameter. The exploration parameter seeks to explore the sequence space (referring to the n-dimensional space representing all possible sequence reads), whereas the exploitation parameter seeks to maximize achievement of an objective. For example, a mutation algorithm that prioritizes the exploitation parameter may seek to identify mutations that maximally increase the binding affinity of the synthetic candidate sequence read. As another example, a mutation algorithm that prioritizes the exploration parameter may seek to choose mutations that differentiate from prior iterations to further explore the sequence space. In one or more embodiments, the synthetic sequence generation module 550 may utilize a diverse set of initial synthetic candidate sequence reads, i.e., to start exploration of the sequence space at spread out positions in the sequence space. This ensures that the synthetic sequence generation module 550 avoids fixating on local optima, rather than exploring for the global optima.

The analytics system may identify one or more optimal candidate sequence reads for manufacturing antibodies for a treatment of a particular disease. The selection of the optimal candidate sequence reads may be according to one or more selection criteria. One example selection criterion selects one or more top-ranked candidate sequence reads, when ranked according to predicted binding affinity. Another example criterion selects the optimal sequence read(s) based on a diversity of sequences of the optimal candidate sequence reads. Diversity is a metric of variation in sequences between a set of sequence reads. For example, if two sequence reads are substantially the same with only a small set of sequences being different between the two sequence reads, then the set of the two sequence reads is of low diversity. On the flip side, if two sequence reads are widely different in their sequences, then the set of the two sequence reads is of high diversity.

The database 560 stores data used by the analytics system 120. Data may include sequencing data from a sequencing system (e.g., the sequencing system 110). In particular, the sequencing data may include sequence reads as output by the sequencing system, e.g., corresponding to nucleic acid fragments coding for candidate antibodies screened in a binding affinity assay. The data may further include information on binding affinities corresponding to the candidate antibodies assessed in the binding affinity assay against a target antigen. In some embodiments, the data may include synthetic candidate sequence reads generated by the synthetic sequence generation module 550, optionally along with predicted binding affinities or binding affinities as measured from a binding affinity assay. The data may further include information gathered from third-party systems 130. The database 560 may further store any of the models sued by the analytics system 120, including but not limited to the binding affinity prediction model, the mutation algorithm, other machine-learning models trained and/or deployed by the analytics system 120, etc.

The advantages of the leveraging the analytics system 120 to aid in candidate antibody exploration are numerous. For one, leveraging a binding affinity prediction model allows the analytics system 120 to make informed predictions from a sparse set of clinical data. For example, the analytics system 120 may utilize sequencing data and binding affinity data from one iteration of a biopanning screening to train the binding affinity prediction model. That initially trained model can serve as a springboard to generating the synthetic candidate sequence reads. In effect, the analytics system can complete candidate antibody exploration with a fraction of clinical experimentation required, thereby saving assaying resources and costs. Furthermore, insight via the interpretability scores better inform the mutation algorithm to generate synthetic candidate sequence reads. Moreover, the synthetic candidate sequence reads may result in higher binding affinities, higher diversity, higher candidate sequence reads of a threshold binding affinity, etc., compared to candidate antibodies identified through traditional screening. In sum, there are numerous advantages to leveraging the analytic system 120 in candidate antibody exploration.

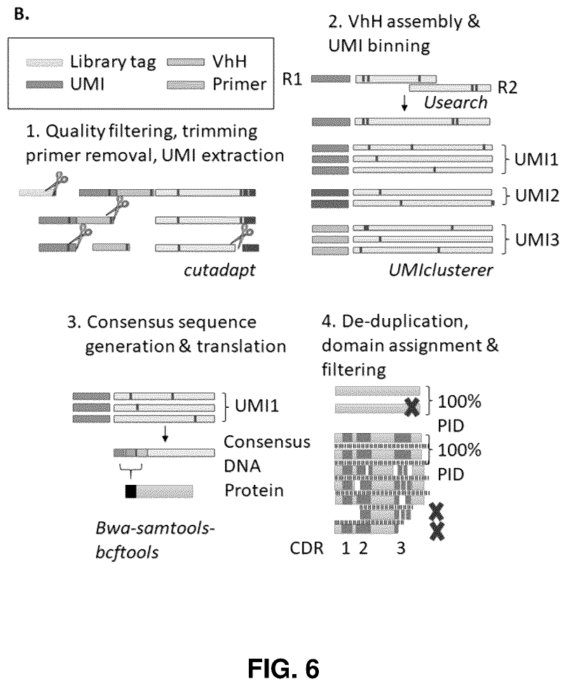

FIG. 6 illustrates processing of sequence reads from a biopanning screening, according to one or more example implementations. The sequencing may be performed by the sequencing system 110 of FIG. 1. Processing and analysis of the sequencing data may be performed by the analytics system 120 of FIG. 1.

Panel B illustrates four steps to processing sequence reads of protein molecules, in preparation for performing analyses with the sequence reads. As background, the sequencing process yields sequence reads of target genes, i.e., nucleic acid molecules in a sample that are used to code for antibodies. The sequence reads, as received by the sequencing system 110, may include sequences pertaining to the target gene, sequences pertaining to a primer used in sequencing, sequences pertaining to a UMI used in sequencing, sequences pertaining to one or more library indices used in sequencing, or some combination thereof.

At step 1, the analytics system 120 performs quality filtering, trimming of sequence reads, primer removal, UMI extraction, or some combination thereof. Quality filtering may involve removing sequence reads outside a range of threshold sequence lengths, removing sequence reads with above a threshold number of indeterminate sequences, removing sequence reads that are due to contamination, etc. Trimming of sequence reads may entail removal of tail ends of sequence reads that may have been ligated to the protein molecules to protect the protein molecule during the sequencing process. Primer removal may entail removal of primers used in the sequencing process. UMI extraction entails identifying the sequences relating to the UMI, which may be located in a known position relative to other sequences in the sequence read (e.g., the UMI is in between the library index and the primer).

At step 2, the analytics system 120 performs VHH assembly, UMI binning, or some combination thereof. VHH assembly comprises combining sequence read pairs to generate a single sequence read pertaining to the nucleic acid molecule. UMI binning entails grouping sequence reads together based on at least their corresponding UMI. Binning may further group based on other properties, e.g., length of sequence reads, similarity of sequence reads, etc. As illustrated in FIG. 6, the analytics system may form at least three bins. In each bin, the sequence reads comprise the same sequence pertaining to the same UMI. Between bins, sequence reads from one bin comprise different sequences compared to sequence reads from another bin, indicating different UMIs between the bins.

At step 3, the analytics system 120 performs consensus sequence generation and translation. Consensus sequence generation entails collapsing sequence reads in a bin into a consensus sequence read. The analytics system may, for each sequence, determine a consensus for that sequence among the sequence reads in the bin. For example, at position 1, the analytics system determines the majority of sequence reads indicates a first nucleotide, hence the first nucleotide is the consensus sequence at position 1. The consensus sequence read may then be translated into expressed amino acid sequences.

At step 4, the analytics system 120 performs de-duplication, domain assignment, filtering, or some combination thereof. De-duplication entails identifying potentially duplicative sequence reads. The analytics system may determine a distance measure between each of the unique sequence reads to determine whether two sequence reads are sufficiently similar to be deemed duplicates. Domain assignment refers to assigning sequences to each complementarity determining regions from a given sequence (e.g., CDRH1, CDRH2, CDRH3).

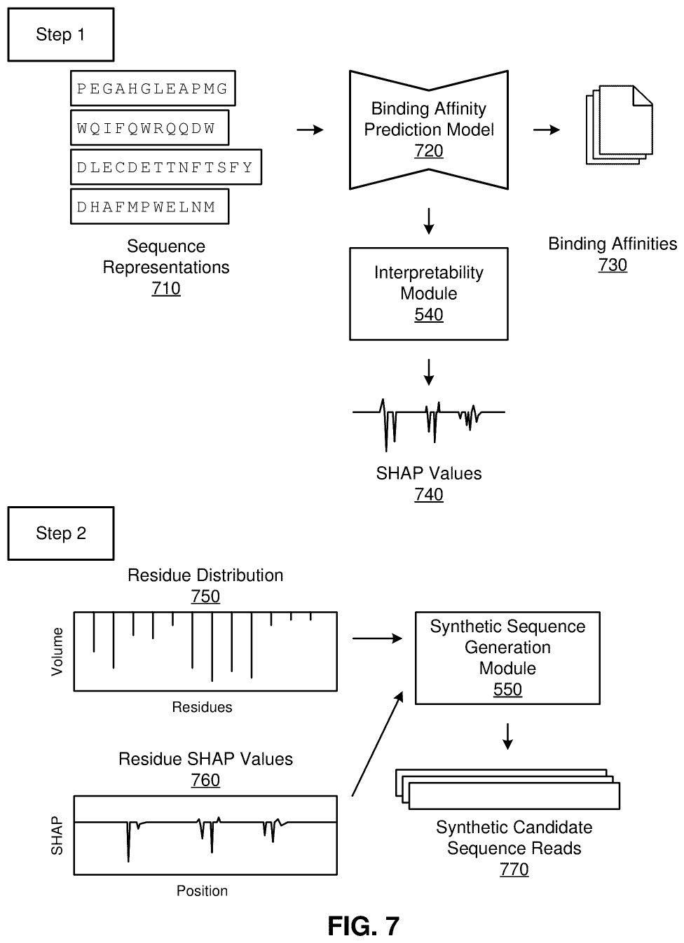

FIG. 7 illustrates generating synthetic sequences leveraging a binding affinity prediction model to interpret binding affinities, according to one or more embodiments. The analytics system 120 may perform some or all of the steps. In other embodiments, other entities may perform some or all of the steps illustrated in FIG. 7. In one or more embodiments, candidate antibody exploration by the analytics system 120 can be separated into two overarching steps.

In step 1, the analytics system 120 trains a binding affinity prediction model that leverages sequencing data and binding affinity data from a clinical assay. The analytics system 120 receives sequence reads from the sequencing system 110. The sequence reads relate to target genes used to code candidate antibodies from a biopanning screening process. The sequence reads may be associated with binding affinities, as determined from a binding affinity assay. The analytics system 120 may perform various preprocessing techniques to prepare the sequence reads for use in training the binding affinity prediction model 720. The analytics system 120 further encodes the sequence reads into sequence representations 710, wherein each sequence representation corresponds to a gene that codes for a unique candidate antibody. The analytics system 120 trains the binding affinity prediction model 720 with the sequence representations 710 and associated binding affinities. The trained binding affinity prediction model 720 may input additional sequence representations 710 to output predicted binding affinities 730. The interpretability module 540 of the analytics system 120 may further generate an interpretability score for each sequence representation. In particular embodiments, the interpretability score includes Shapley (SHAP) values 740 for the features in the sequence representations 710.

In step 2, the analytics system 120 generates synthetic candidate sequence reads 770 leveraging the binding affinity prediction model 720 and the interpretability scores determined by the interpretability module 540. The synthetic sequence generation module 550 inputs a residue distribution 750 and residue Shapley (SHAP) values 760 to output synthetic candidate sequence reads 770. The residue distribution 750 can inform occurrence with which residues have come up in candidate sequence reads. The residue distribution 750 may be paired with residue SHAP values 760. Each residue represented in the residue distribution 750 may have a corresponding SHAP value in the residue SHAP values 760. The SHAP value may be derived from a single SHAP value from an interpretability score for a single sequence read, or may be an average of SHAP values from interpretability scores for a plurality of sequence reads with the residue. The synthetic candidate sequence reads 770 output by the synthetic sequence generation module 550 may be input into the binding affinity prediction model 720 to predict a binding affinity for each synthetic candidate sequence read.

The synthetic sequence generation module 550 may perform further iterations of synthetic sequence generation. For example, based on the predicted binding affinities for the synthetic candidate sequence reads 770, the synthetic sequence generation module 550 may generate a new set of synthetic candidate sequence reads that are different from a first set of synthetic candidate sequence reads. The new set of synthetic candidate sequence reads may be generated based on the first set (and/or other prior sets). For example, a synthetic candidate sequence read may be mutated to generate a new synthetic candidate sequence read. The analytics system 120 may continue iterating until certain objectives are achieved. With the synthetical candidate sequence reads, the analytics system 120 may select one or more of them to be part of a treatment. The analytics system 120 may further provide one or more of the synthetic candidate sequence reads for manufacturing candidate antibodies to be assessed in a binding affinity assay.

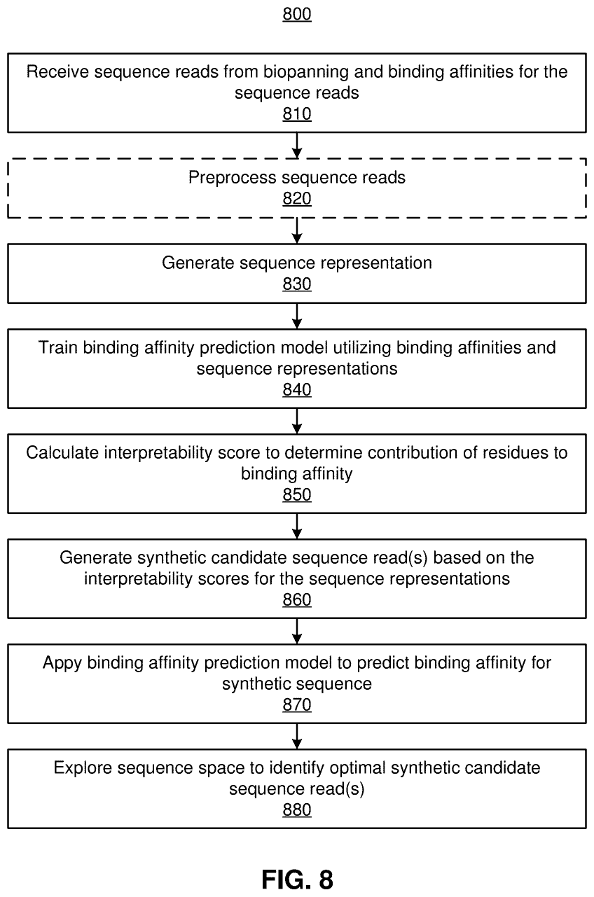

FIG. 8 is a flowchart of a method of antibody affinity tuning, according to one or more embodiments. The method 800 is described as being performed by the analytics system 120, but, in other embodiments, some or all of the steps in the method 800 may be performed by other entities in the computing environment 100 or other computing devices. In other embodiments, there may be additional steps, fewer steps, different steps, or steps in a different order as part of the method 800.

The analytics system 120 receives 810 sequence reads from a biopanning screening and binding affinities for the sequence reads. The biopanning screening creates a library of candidate antibodies which are assessed for their binding affinity to a target antigen via a binding affinity assay. Candidate antibodies above a threshold binding affinity may be selected for sequencing by a sequencing system. In one or more embodiments, the sequence reads pertain to target genes that code for the candidate antibodies. The sequence reads output by the sequencing system may include sequences pertaining to a primer, sequences pertaining to one or more library indices, sequences pertaining to a unique molecule identifier (UMI), sequences pertaining to the target gene, or some combination thereof.

The analytics system 120 may preprocess 820 the sequence reads, e.g., in preparation for downstream analyses. The analytics system may preprocess the sequence reads by demultiplexing sequence reads from the sequencing system. Demultiplexing relies on separating sequence reads based on one or more library indices. Sequence reads pertaining to one sample are further processed to remove sequences relating to primers, along with other quality filtering techniques. The sequence reads are also binned by UMI. With a bin of sequence reads, generally pertaining to the same sequenced molecule, the analytics system 120 may collapse the sequence reads into a consensus sequence read. In embodiments where the sequence reads describe nucleotides, the analytics system 120 may translate the nucleotide sequences into amino acid sequences.

The analytics system 120 generates 830 sequence representations for the sequence reads. The sequence representation is a vector of features. Each sequence read may have different values for the features in its sequence representation, compared to other sequence reads. The sequence representation may include abstract features relating to other chemical properties, other physical properties, or any other characteristics of the sequence read. In one or more embodiments, each feature is a residue, comprising one or more amino acid sequences.

The analytics system 120 trains 840 a binding affinity prediction model utilizing the sequence representations and the binding affinities. The binding affinity prediction model inputs a sequence representation and outputs a predicted binding affinity. The analytics system 120 may train the binding affinity prediction model as supervised machine-learning model.

The analytics system 120 calculates 850 an interpretability score to determine contribution of residues to the predicted binding affinity for a sequence representation. The interpretability score may include Shapley (SHAP) values for the features (e.g., the residues) of a sequence representation. The interpretability score indicates how each feature contributed to the binding affinity prediction. The contribution can indicate positive or negative and may also indicate magnitude of the contribution.

The analytics system 120 generates 860 one or more synthetic candidate sequence reads based on the interpretability scores for the sequence representations. The analytics system 120 generates one or more synthetic candidate sequence reads based on the interpretability scores for the sequence representations. The analytics system 120 may generate a synthetic candidate sequence read randomly, e.g., random amino acid sequences. In other embodiments, the analytics system 120 may utilize a prior candidate sequence read as a base to mutate one or more amino acid sequences, or one or more residues. In other embodiments, the analytics system 120 may combine residues from different candidate sequence reads to generate a synthetic candidate sequence read.

The analytics system 120 applies 870 the binding affinity prediction model to predict a binding affinity for a sequence representation for a synthetic candidate sequence read. With the synthetic candidate sequence read, the analytics system 120 may generate a sequence representation. The binding affinity prediction model is applied to the sequence representation to predict a binding affinity. The predicted binding affinity may be a discrete value or a value selected from a continuous range.

The analytics system 120 explores 880 the sequence space to identify optimal synthetic candidate sequence read(s). The analytics system 120 may further explore the sequence space by iteratively generating new synthetic candidate sequence reads based on predicted binding affinities of previously generated synthetic candidate sequence reads. The analytics system 120 may explore the sequence space utilizing one or more optimization algorithms. Such algorithms can balance exploration and exploitation to generate newer synthetic candidate sequence reads.

Further, the analytics system 120 may assess the synthetic candidate sequence reads in a binding affinity assay. In one or more embodiments, the analytics system 120 may provide the synthetic candidate sequence reads to a manufacturing system to express candidate antibodies to assess binding affinity of those candidate antibodies, e.g., in a binding affinity assay. The analytics system 120 may further leverage binding affinity data from the binding affinity assay to refine the binding affinity prediction model, the mutation algorithm implemented during synthetic sequence generation, the optimization algorithm, or some combination thereof.

Further, the analytics system 120 may assemble optimal synthetic candidate sequence reads into a treatment for a particular disease. The analytic system 120 may assemble optimal synthetic candidate sequence reads into a treatment based on one or more selection criteria. For example, the analytics system 120 may identify synthetic candidate sequence reads with the highest binding affinity to be used as part of the treatment. In other examples, the analytics system 120 may select synthetic candidate sequence reads with a threshold binding affinity strength but with diversity between the synthetic candidate sequence reads.

### Computer Architecture

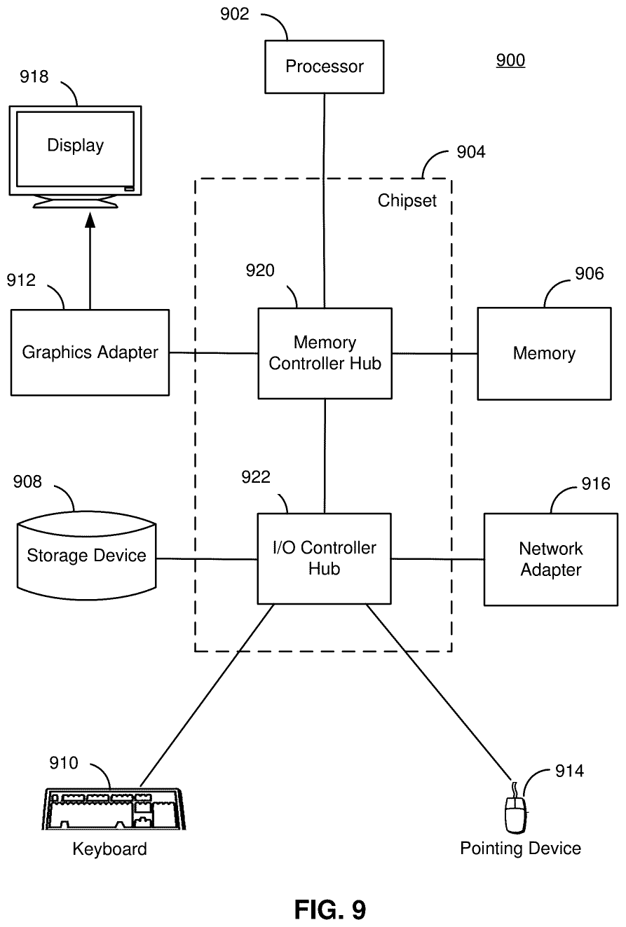

FIG. 9 is a block diagram of an example computer 900, in accordance with one or more embodiments. The example computer 900 includes one or more processors 902 coupled to a chipset 904. The chipset 904 includes a memory controller hub 920 and an input/output (I/O) controller hub 922. A memory 906 and a graphics adapter 912 are coupled to the memory controller hub 920, and a display 918 is coupled to the graphics adapter 912. A storage device 908, keyboard 910, pointing device 914, and network adapter 916 are coupled to the I/O controller hub 922. Other embodiments of the computer 900 have different architectures.

In the embodiment shown in FIG. 9, the storage device 908 is a non-transitory computer-readable storage medium, for example, such as a hard drive, compact disk read-only memory (CD-ROM), DVD, or a solid-state memory device. The memory 906 holds instructions and data used by the processor 902. The pointing device 914 is a mouse, track ball, touch-screen, or other type of pointing device, and may be used in combination with the keyboard 910 (which may be an on-screen keyboard) to input data into the computer system 900. The graphics adapter 912 displays images and other information on the display 918. The network adapter 916 couples the computer system 900 to one or more computer networks, such as network 150. The types of computers used by the entities of FIG. 1 can vary depending upon the embodiment and the processing power required by the entity. Furthermore, the computers can lack some of the components described above, such as keyboards 910, graphics adapters 912, and displays 918.

### Additional Considerations

Some portions of above description describe the embodiments in terms of algorithmic processes or operations. These algorithmic descriptions and representations are commonly used by those skilled in the computing arts to convey the substance of their work effectively to others skilled in the art. These operations, while described functionally, computationally, or logically, are understood to be implemented by computer programs comprising instructions for execution by a processor or equivalent electrical circuits, microcode, or the like. Furthermore, it has also proven convenient at times, to refer to these arrangements of functional operations as modules, without loss of generality.

As used herein, any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Similarly, use of “a” or “an” preceding an element or component is done merely for convenience. This description should be understood to mean that one or more of the elements or components are present unless it is obvious that it is meant otherwise.

Where values are described as “approximate” or “substantially” (or their derivatives), such values should be construed as accurate +/−10% unless another meaning is apparent from the context. From example, “approximately ten” should be understood to mean “in a range from nine to eleven.”

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Upon reading this disclosure, those of skill in the art will appreciate still additional alternative structural and functional designs. Thus, while particular embodiments and applications have been illustrated and described, it is to be understood that the described subject matter is not limited to the precise construction and components disclosed. The scope of protection should be limited only by any claims that may ultimately issue.

What is claimed is:

1. A computer-implemented method comprising:
receiving sequence reads from a sequencing system, the sequence reads associated with binding affinities to a target antigen, wherein each sequence read comprises a target gene for expressing a candidate antibody;
generating sequence representations from the sequence reads, wherein each sequence representation is a feature vector representing the amino acid sequences of a corresponding sequence read;
training a binding affinity prediction model with the sequence representations and the binding affinities, wherein the binding affinity prediction model is configured to input a sequence representation and to output a binding affinity prediction;
generating a synthetic candidate sequence read that is different from the sequence reads;
generating a sequence representation for the synthetic candidate sequence read; and
determining a binding affinity prediction for the synthetic candidate sequence read by applying the binding affinity prediction model to the sequence representation for the synthetic candidate sequence read.

2. The computer-implemented method of claim 1, further comprising:
preprocessing the sequence reads, prior to generating the sequence representation for each sequence read.

3. The computer-implemented method of claim 2, wherein preprocessing the sequence reads comprises:
demultiplexing the sequence reads by grouping matching sequences relating to one or more library indices for each of the sequence read into one sample.

4. The computer-implemented method of claim 2, wherein preprocessing the sequence reads comprises:
removing one or more sequences pertaining to a primer used in sequencing of the target gene.

5. The computer-implemented method of claim 2, wherein preprocessing the sequence reads comprises:
identifying, for each sequence read, sequences relating to one unique molecule identifier of a plurality of unique molecule identifiers;
grouping sequence reads of similar length and having the same unique molecule identifier in a bin; and
collapsing the sequence reads in each bin into a consensus sequence read.

6. The computer-implemented method of claim 2, wherein preprocessing the sequence reads comprises:
assembling each sequence read from a forward sequence read and a reverse sequence read of one nucleic acid molecule.

7. The computer-implemented method of claim 2, wherein preprocessing the sequence reads comprises:
translating a sequence read from nucleotide sequences into amino acid sequences.

8. The computer-implemented method of claim 1, wherein the binding affinity prediction model is a machine-learning model.

9. The computer-implemented method of claim 8, wherein the machine-learning model is one of a deep-learning neural network, a convolutional neural network, or a residual neural network.

10. The computer-implemented method of claim 1, wherein generating the synthetic candidate sequence read comprises:
identifying a base sequence read with a high binding affinity; and
mutating one or more amino acid sequences of the base sequence read.

11. The computer-implemented method of claim 1, further comprising:
calculating an interpretability score for each sequence representation that indicates the contribution of each feature in a given sequence representation to the predicted binding affinity for the given sequence representation.

12. The computer-implemented method of claim 11, wherein the interpretability score comprises a Shapley value for each feature of the sequence representation.

13. The computer-implemented method of claim 1, further comprising:
determining the synthetic candidate sequence read is fit for a treatment of a particular disease if the binding affinity prediction for the synthetic candidate sequence read is above a threshold.

14. The computer-implemented method of claim 13, further comprising:
instructing a manufacturing system to manufacture the treatment for the particular disease comprising antibodies expressed from the synthetic candidate sequence read.

15. The computer-implemented method of claim 1, further comprising:
performing an optimization algorithm comprising iteratively:
generating a subsequent synthetic candidate sequence read,
determining a binding affinity prediction for the subsequent synthetic candidate sequence read by applying the binding affinity prediction model to a sequence representation for the subsequent synthetic candidate sequence read, and
evaluating whether the binding affinity prediction improves upon a prior binding affinity prediction for a prior synthetic candidate sequence read.

16. A non-transitory computer-readable storage medium storing instructions that, when executed by one or more processors, cause the one or more processors to:
receive sequence reads from a sequencing system, wherein each sequence read comprises a target gene for expressing a candidate antibody, wherein each sequence read is associated with a binding affinity to a target antigen;
generate a sequence representation for each sequence read, wherein the sequence representation is a feature vector representing the amino acid sequences of the sequence read;
train a binding affinity prediction model with the sequence representations and the binding affinities, wherein the binding affinity prediction model is configured to input a sequence representation and to output a binding affinity prediction;
generate a synthetic candidate sequence read that is different from the sequence reads;
generate a sequence representation for the synthetic candidate sequence read; and
determine a binging affinity prediction for the synthetic candidate sequence read by applying the binding affinity prediction model to the sequence representation for the synthetic candidate sequence read.

17. The non-transitory computer-readable storage medium of claim 16, the instructions further causing the one or more processors to:
preprocess the sequence reads, prior to generating the sequence representation for each sequence read.

18. The non-transitory computer-readable storage medium of claim 17, wherein to preprocess the sequence reads comprises to:
demultiplex the sequence reads by grouping matching sequences relating to one or more library indices for each of the sequence read into one sample.

19. The non-transitory computer-readable storage medium of claim 17, wherein to preprocess the sequence reads comprises to:
remove one or more sequences pertaining to a primer used in sequencing of the target gene.

20. The non-transitory computer-readable storage medium of claim 17, wherein to preprocess the sequence reads comprises to:
identify, for each sequence read, sequences relating to one unique molecule identifier of a plurality of unique molecule identifiers;
group sequence reads of similar length and having the same unique molecule identifier in a bin; and
collapse the sequence reads in each bin into a consensus sequence read.

21. The non-transitory computer-readable storage medium of claim 17, wherein to preprocess the sequence reads comprises to:
assemble each sequence read from a forward sequence read and a reverse sequence read of one nucleic acid molecule.

22. The non-transitory computer-readable storage medium of claim 17, wherein to preprocess the sequence reads comprises to:
translate a sequence read from nucleotide sequences into amino acid sequences.

23. The non-transitory computer-readable storage medium of claim 16, wherein the binding affinity prediction model is a machine-learning model.

24. The non-transitory computer-readable storage medium of claim 23, wherein the machine-learning model is one of a deep-learning neural network, a convolutional neural network, or a residual neural network.

25. The non-transitory computer-readable storage medium of claim 16, wherein to generate the synthetic candidate sequence read comprises to:
identify a base sequence read with a high binding affinity; and
mutate one or more amino acid sequences of the base sequence read.

26. The non-transitory computer-readable storage medium of claim 16, the instructions further causing the one or more processors to:
calculating an interpretability score for each sequence representation that indicates the contribution of each feature in a given sequence representation to the predicted binding affinity for the given sequence representation.

27. The non-transitory computer-readable storage medium of claim 26, wherein the interpretability score comprises a Shapley value for each feature of the sequence representation.

28. The non-transitory computer-readable storage medium of claim 16, the instructions further causing the one or more processors to:
determine the synthetic candidate sequence read is fit for a treatment of a particular disease if the binding affinity prediction for the synthetic candidate sequence read is above a threshold.

29. The non-transitory computer-readable storage medium of claim 28, the instructions further causing the one or more processors to:
instruct a manufacturing system to manufacture the treatment for the particular disease comprising antibodies expressed from the synthetic candidate sequence read.

30. The non-transitory computer-readable storage medium of claim 16, the instructions further causing the one or more processors to:
perform an optimization algorithm comprising to iteratively:
generate a subsequent synthetic candidate sequence read,
determine a binding affinity prediction for the subsequent synthetic candidate sequence read by applying the binding affinity prediction model to a sequence representation for the subsequent synthetic candidate sequence read, and
evaluate whether the binding affinity prediction improves upon a prior binding affinity prediction for a prior synthetic candidate sequence read.
